(19) United States
(12) Patent Application Publication
(10) Pub. No.: US 2007/0043438 A1
(43) Pub. Date: Feb. 22, 2007

(54) BONE AND LIGAMENT GRAFT MODIFICATION SYSTEM

(75) Inventor: Mark A. Feanny, Pearland, TX (US)

Correspondence Address: CONLEY ROSE, P.C., P. O. BOX 3267, HOUSTON, TX, 77253-3267, US

(73) Assignee: BAYLOR COLLEGE OF MEDICINE, Houston, TX (US)

(21) Appl. No.: 11/457,522

(22) Filed: Jul. 14, 2006

Related U.S. Application Data
(60) Provisional application No. 60/595,557, filed on Jul. 14, 2005.

(51) Int. Cl. A61F 2/08

(52) U.S. Cl. 623/13.14

(57) ABSTRACT

Methods and apparatus for modifying the bone portion of a graft, such as that used in the reconstruction of a damaged anterior cruciate ligament. Embodiments of the present invention comprise a driving member for threaded engagement with a housing member and a cutting member engaged with the driving member. The housing member is capable of receiving and supporting the bone graft without damaging a ligament attached thereto. Upon rotation of the driving member, the cutting member will engage the bone portion of the graft and modify it to have a generally circular cross section.

### CROSS-REFERENCE TO RELATED APPLICATIONS

This application claims benefit of U.S. provisional application Ser. No. 60/595,557, filed Jul. 14, 2005, and entitled “Bone and Ligament Graft Modification System,” which is incorporated by reference in its entirety

### STATEMENT REGARDING FEDERALLY SPONSORED RESEARCH OR DEVELOPMENT

Not Applicable

### BACKGROUND INFORMATION

Embodiments of the present invention are directed toward methods and apparatus for modifying a bone graft to have a generally circular cross section. Embodiments of the present invention are particularly directed towards methods and apparatus for modifying the bone section of a bone and ligament graft used in the reconstruction of a damaged ligament, such as the anterior cruciate ligament (ACL). Although the procedure and associated problems of ACL reconstructive surgery are described below, embodiments of the present invention may be used in other procedures where it is desired to produce bone grafts that have a generally circular cross section and are generally cylindrical in shape.

In a typical ACL reconstructive surgery, a graft is harvested from each of the patellar and tibial bones. As harvested, these patellar and tibial bone grafts are connected by a tendinous attachment, which will be used to reconstruct the damaged ACL ligament. When harvested, the bone grafts are typically not cylindrical in shape and do not have a circular cross section. However, as part of the reconstruction/replacement of the ACL, the grafts will be inserted into generally circular holes drilled into the femur and tibia. Therefore, the grafts need to be re-shaped to conform to the recipient holes and provide a proper fit between the graft and the hole. A surgeon typically shapes the bone graft with a rongeur by removing a small amount of material and then checking the graft against a gauge or other sizing device.

The surgeon uses trial and error to shape the bone graft to conform to the gauge, which is approximately the same size as the hole that will be drilled in the recipient bone. This trial and error process can require considerable time and extend the length of the operation, increasing both the cost of surgery and the potential risks to the patient. The surgeon must also take care not to remove too much material from the graft, which would result in a loose fit between the graft and recipient bone. A loose fit between the graft and recipient bone increases the likelihood that the graft will not properly heal and could result in the graft being pulled out of the recipient bone completely. The use of a rongeur also requires extensive handling of the graft, which increases the risk of bacterial infection or possibly even dropping the graft.

Some prior art devices, such as that disclosed in U.S. Pat. No. 6,776,938 (incorporated herein by reference), have attempted to address the issues described above. However, such prior art devices potentially create other issues by generating potentially damaging compressive and shear forces on the bone graft.

Hence, it remains desirable to provide a method and apparatus for modifying a bone graft that does not require excessive time or necessitate extensive handling of the graft. It is further desired to provide a method and apparatus for modifying a bone graft that creates a uniform or standard size and shape of the graft without subjecting the graft to potentially damaging forces.

### SUMMARY OF THE PREFERRED EMBODIMENTS

Embodiments of the present invention relate to methods and apparatus for modifying the bone cross section of a bone portion of a graft, such as that used in the reconstruction of a damaged anterior cruciate ligament (ACL). Embodiments of the present invention comprise a driving member for threaded engagement with a housing member and a cutting member engaged with the driving member. Upon rotation of the driving member, the cutting member will engage the bone portion of the graft and modify it to have a generally circular cross section.

### BRIEF DESCRIPTION OF THE DRAWINGS

FIG. 1 is a view of a disassembled graft modification system;

FIG. 2 is a perspective view of the housing member of the graft modification system of FIG. 1;

FIG. 3 is an assembled view of the graft modification system of FIG. 1;

FIG. 4 is a view of an unmodified graft;

FIG. 5 is a view of the graft and graft modification system;

FIG. 6 is a view of a modified graft.

### DETAILED DESCRIPTION OF THE PREFERRED EMBODIMENTS

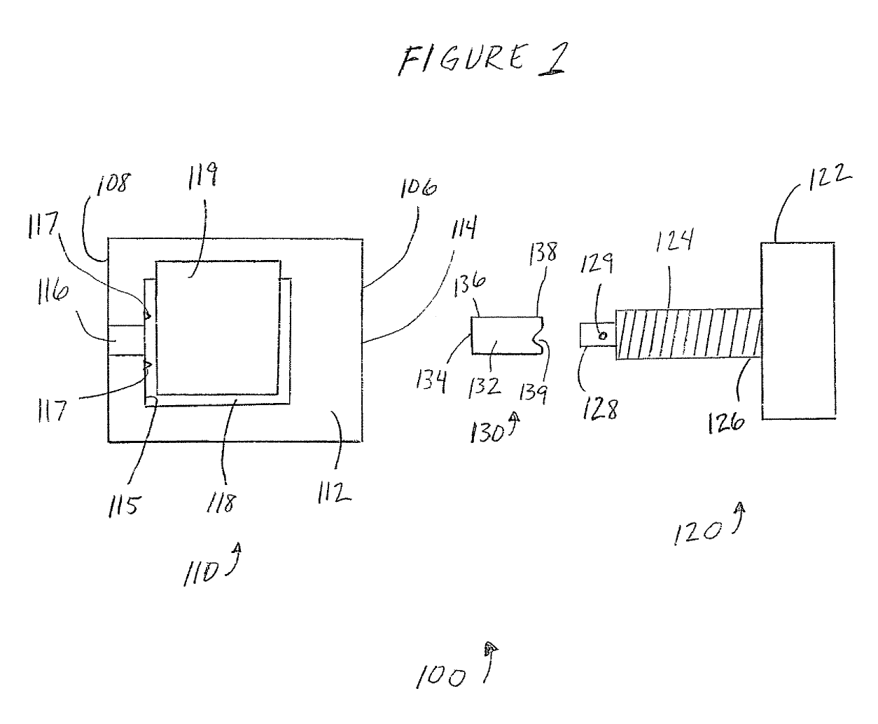

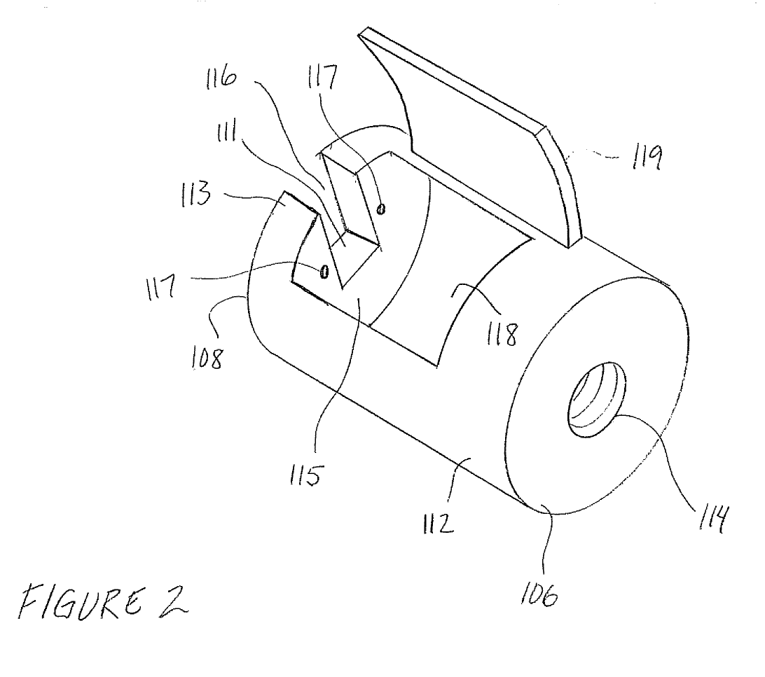

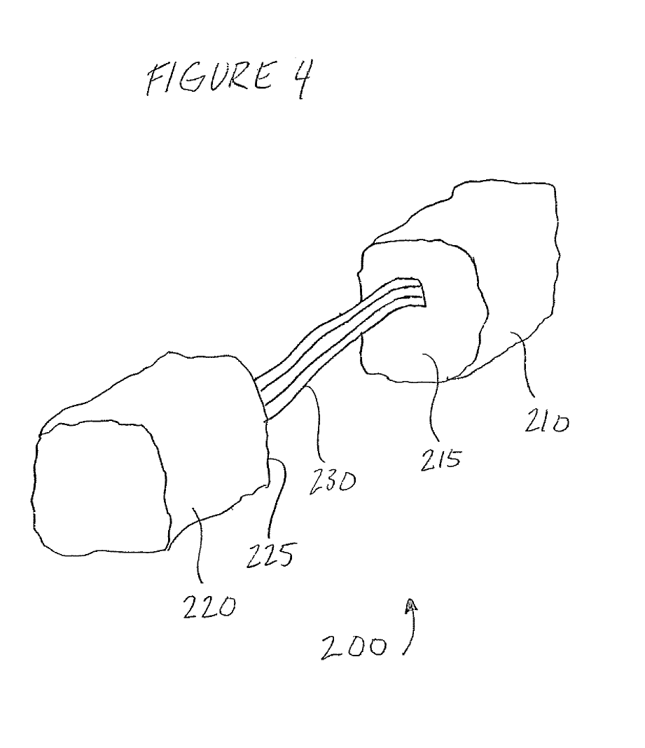

Referring initially to FIGS. 1-3, a graft modification system (GMS) 100 for, modifying a bone/ligament graft 200 (shown in FIG. 4) is displayed. As shown in the unassembled view of the embodiment in FIG. 1, GMS 100 comprises a housing member 110, a driving member 120, and a cutting member 130. As best shown in the perspective view of FIG. 2, housing member 110 comprises a generally cylindrical body 112 having an outer surface 113 and an recess 118 defined between a drive end wall 106 and a graft end wall 108. In the embodiment shown, drive end 106 wall has a threaded bore 114 therethrough for receiving driving member 120, while graft end wall 108 comprises an opening or slot 116. An openable cover member 119 allows or prevents access to recess 118. The graft end of recess 118 is defined by an inner wall 115 having a pair of anti-rotation members 117 protruding inwardly therefrom. In the embodiment shown, slot 116 extends radially inwardly from outer surface 113 to an inner portion 111 of graft end wall 108.

Referring back to FIG. 1, driving member 120 comprises a threaded shaft 124 with a handle 122 disposed on a distal end 126 and a pin or engagement member 129 disposed on a proximal end 128. In the embodiment shown, cutting member 130 comprises a tubular cylindrical body portion 132 having a cutting edge 134 on a first end 136 and a slot or engagement member 139 disposed on a second end 138.

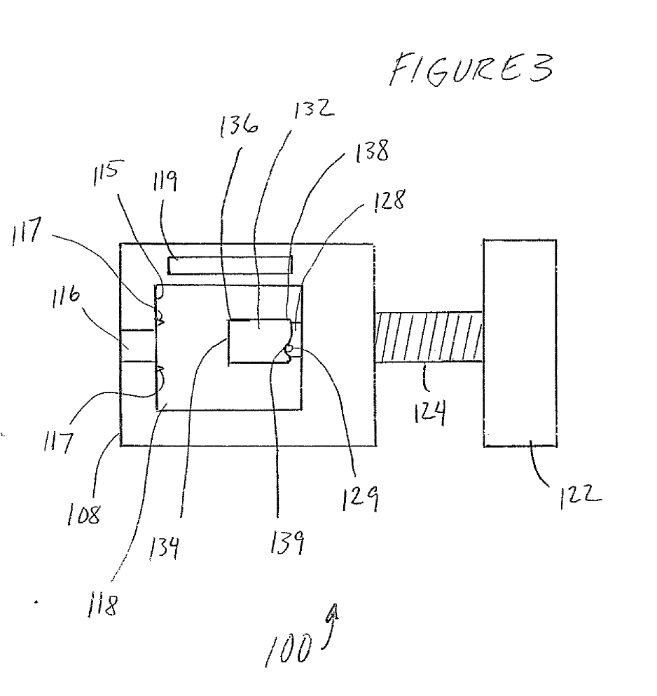

In the assembled view of the embodiment in FIG. 3, cutting member 130 is disposed on proximal end 128 of threaded shaft 124, such that engagement members 129 and 139 are engaged. In addition, threaded shaft 124 is threadably engaged with threaded bore 114, so that cutting member 130 is disposed within recess 118.

As shown in FIG. 4, a bone/ligament graft 200 comprises a first bone portion 210 harvested from a first bone and a second bone portion 220 harvested from a second bone. Prior to harvest, first and second bone portions 210, 220 are selected from bones that are connected by ligament. Sections of bone surrounding the connection points at each end of the ligament are harvested, so that the ligament 230 still connects them after, harvest. First and second bone portions 210 and 220 each have an inner end 215, 225, respectively, which are connected to ligament portion 230. As described in the previous discussion of ligament transplants, the bone portions of the graft will be inserted into generally circular holes that have been drilled into the recipient bones (not shown). However, first and second bone portions 210 and 220 are initially irregularly shaped because they are typically removed from the donor bones (not shown) by chiseling or other methods that typically produce irregular shaped portions. As previously described, it is desirable for the bone grafts to have a fairly tight fit into the holes of the recipient bones and to maximize the contact between the grafts and the recipient bones. Therefore, first and second bone portions 210 and 220 typically need to be modified to have a generally circular cross section so as to ensure a proper fit into the holes in the recipient bones.

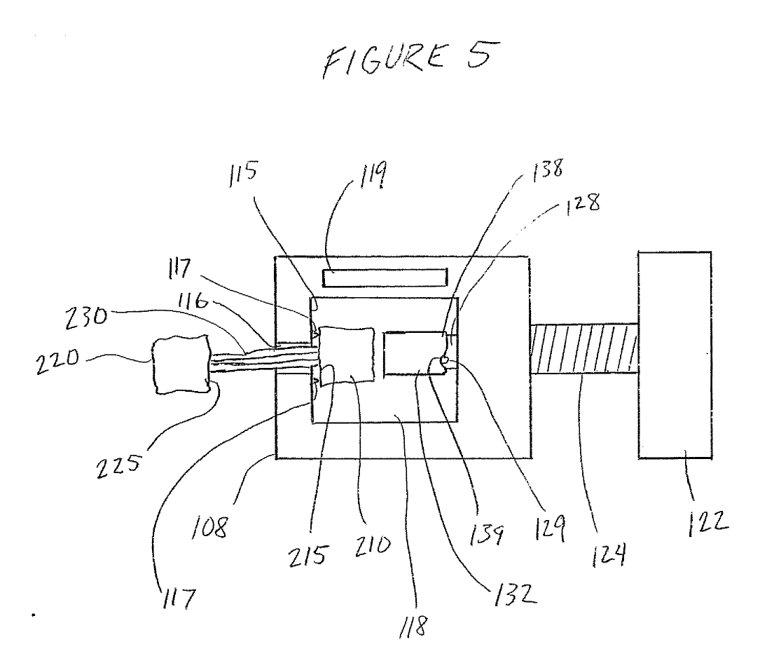

In the view of FIG. 5, cutting member 130 is engaged with driving member 120 and threaded shaft 124 is threadably engaged with threaded bore 114, so that cutting member 130 is disposed within recess 118. Also shown in FIG. 5, first bone portion 210 has been placed in recess 118 with ligament portion 230 disposed within slot 116 of housing member 110. In the embodiment shown, anti-rotation members 117 are sharp protuberances that extend from inner wall 115 and contact inner end 215 of first bone portion 210, as explained more fully below. After first bone portion 210 has been placed in recess 118, cover member 119 is closed to capture bone portion 210 within recess 118. In the embodiment shown, GMS 100 is operated by rotating handle 122 clockwise so that threaded shaft 124 engages threaded bore 114 and advances cutting member 130 towards bone portion 210. After cutting member 130 has moved a sufficient axial distance, cutting surface 134 will engage first bone portion 210.

Due to the threaded engagement of threaded shaft 124 and threaded bore 114, cutting edge 134 will rotate as it moves axially towards first bone portion 210. In the embodiment shown, cutting surface 134 is a circular sharp edge disposed on first end 136 of cutting member 130. Therefore, as cutting surface 134 rotates and moves axially along bone portion 210, it will cut first bone portion 210 into a generally circular cross section and tubular cylindrical body portion 132 will be disposed around the modified first bone portion 210. Anti-rotation members 117 help to hold first bone portion 210 in place so that first bone portion 210 will not rotate when cutting surface 134 acts on it. In other embodiments, cover member 119 can be configured to contact first bone portion 210 so that it also restricts rotation of first bone portion 210 when cutting surface 134 acts on it. Still other embodiments may combine a combination of anti-rotation devices such as anti-rotation members 117 and cover member 119.

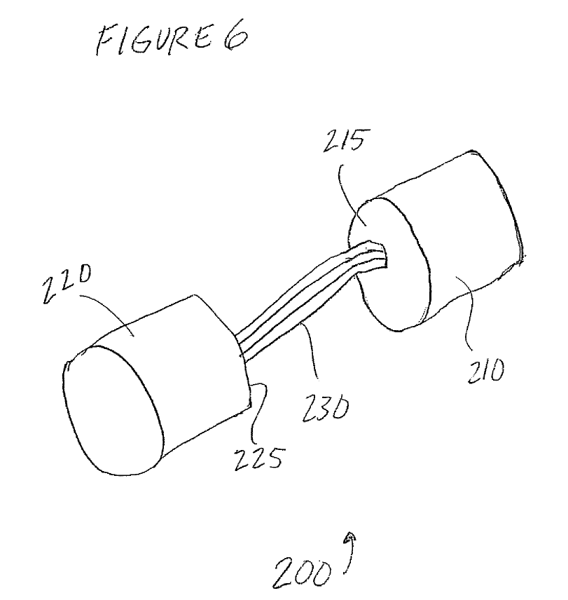

Handle 122 is rotated until cutting surface 134 passes through the length of bone portion 210 and modifies first bone portion 210 to have a generally circular cross section. At that point, the rotation of handle 122 is reversed so that cutting member 130 moves in the opposite axial direction and away from first bone portion 210. Cover 119 is then opened and bone/ligament graft 200 is removed and repositioned so that second bone portion 220 is received within recess 118. The process described above is repeated so that second bone portion 220 is modified to have a generally circular cross section. At this point, first and second bone portions 210 and 220 of bone/ligament graft 200 have each been modified to have a generally circular cross section, as shown in FIG. 6. This allows first and second bone portions 210 and 220 to be installed in the holes of the recipient bones (not shown) with a proper fit and allows more contact between the bone portions 210 and 220 and the recipient bones. This in turn improves healing and ultimate graft strength.

Embodiments of the present invention as described above provide many benefits during surgical procedures such as ligament transplants or reconstructions. For example, embodiments of the invention reduce the amount of time needed to create a generally cylindrical bone portion, resulting in lower costs and decreasing the likelihood of complications during surgery. Time savings are realized by engaging a cutting surface that is distributed around the circumference of the bone (rather than just one portion, as with a rongeur). Time savings are also realized by producing a uniform graft size, minimizing the need to perform trial and error fittings between the graft and a standard gauge used to measure the size of the graft. Producing a uniform graft size also allows for a better fit between the graft and the recipient bone. In addition, embodiments of the present invention allow a surgeon to modify a bone graft without holding the graft in his or her hands. This reduces the likelihood of bacterial contamination of the graft and also minimizes the chance that the graft could be inadvertently dropped, rendering the graft unusable. Furthermore, embodiments of the invention distribute the cutting forces around the circumference of the graft, reducing the likelihood that the bone graft will be damaged during the shaping process.

While various preferred embodiments of the invention have been shown and described, modifications thereof can be made by one skilled in the art without departing from the spirit and teachings of the invention. The embodiments described herein are exemplary only, and are not limiting. Many variations and modifications of the invention and apparatus disclosed herein are possible and are within the scope of the invention. For example, the opening in the graft end of the housing member may be a hole rather than a slot. Other embodiments may also comprise a cutting surface that does not have a completely circular cross section, so long as the cutting surface produces a generally circular bone graft when rotated about the graft. The cutting surface may include teeth, serrations, or other means for enhancing cutting, such as embedded diamond particles. In addition, the cutting member may engage with the driving member in various manners, such as a threaded connection or other suitable mechanical engagement. Other embodiments may also comprise different means for rotating the threaded shaft, such as a wrench. Accordingly, the scope of protection is not limited by the description set out above, but is only limited by the claims that follow, that scope including all equivalents of the subject matter of the claims.

What is claimed is:

1. A system for modifying a bone graft comprising:
a housing member having a first end wall and a second end wall and a recess therebetween, wherein said first end wall comprises a threaded bore therethrough and said second end includes an opening therethrough and allowing access to said recess;
a driving member comprising a threaded portion for threaded engagement with said threaded bore, said threaded portion having a proximal end and a distal end; mid
a cutting member disposed on said proximal end of said threaded portion.

2. The system of claim 1, further comprising:
an openable cover member for securing the bone graft in said recessed portion of said housing member.

3. The system of claim 1 wherein said cutting member extends into said recessed portion of said housing member.

4. The system of claim 1, further comprising an anti-rotation member for preventing rotation of the bone graft, wherein said anti-rotation member extends into said recess.

5. The system of claim 1 wherein said cutting member includes a generally circular cutting edge.

6. The system of claim 1 wherein said driving member further comprises a handle capable of manual rotation by a human operator.

7. The system of claim 1 wherein said opening is a slot extending radially from an outer surface of said second end wall to an interior portion of said second end wall.

8. A system for modifying a bone graft connected to a tendon or ligament, said system comprising:
an elongate body having first and second ends and a recessed portion disposed therebetween, wherein said first end has a threaded bore therethrough and said second end has a slot therethrough and wherein said recessed portion is capable of receiving the bone graft while the tendon or ligament is disposed within said slot;
a threaded shaft with a proximal and a distal end;
means for rotating said threaded shaft disposed on said distal end of said threaded shaft; and
a cutting member disposed on said proximal end of said threaded shaft, wherein said cutting member is disposed within said recessed portion when said threaded shaft is engaged with said threaded bore.

9. The system of claim 8 wherein said means for rotating said threaded shaft is a handle.

10. The system of claim 8 wherein said recessed portion comprises an inner wall substantially perpendicular to said threaded hole and said inner wall includes at least one anti-rotation member projecting into said recessed portion.

11. The system of claim 10 wherein said anti-rotation member comprises a sharp protuberance that engages the bone graft when the bone graft is placed within the recessed portion.

12. The system of claim 8, further comprising a cover for said recessed portion, wherein said cover can be opened to allow a user to place the bone graft in said recessed portion and wherein said cover can be closed to secure the bone graft in said recessed portion.

13. The system of claim 8 wherein the cutting member comprises a generally circular sharp edge.

14. The system of claim 8 wherein said proximal end of said threaded shaft comprises a pin that engages a slot on said cutting member.

15. A method of modifying a bone graft connected to a tendon or ligament, comprising the steps of:
providing a system comprising:
a housing member with a recessed portion between a first end and a second end, wherein said first end comprises a threaded bore and said second end comprises an opening connected to said recessed portion;
a driving member comprising a threaded portion for threaded engagement with said threaded bore, wherein said threaded portion comprises a proximal end and a distal end; and
a cutting member disposed on said proximal end of said threaded portion;
placing the bone graft in said recessed portion such that the tendon or ligament is disposed within said slot;
engaging said threaded portion of said driving member with said threaded bore such that said cutting member is disposed within said recessed portion;
rotating said threaded portion of said driving member so that said cutting member moves toward said bone graft; and
engaging said bone graft with said cutting member so that said cutting member modifies said bone graft to have a generally circular cross section.

16. The method of claim 15, further comprising the steps of:
providing an anti-rotation member in said recessed portion, wherein said anti-rotation member engages the bone graft and restricts the rotational movement of the bone graft when said cutting member engages the bone graft.
